(19) United States
(12) Patent Application Publication
(10) Pub. No.: US 2015/0126711 A1
(43) Pub. Date: May 7, 2015

(54) Method For Obtaining Plant Proteins

(71) Applicants: Thomas Lehmann, Langenselbold (DE); Oskar Lichner, Langenselbold (DE)

(72) Inventors: Thomas Lehmann, Langenselbold (DE); Oskar Lichner, Langenselbold (DE)

(21) Appl. No.: 14/397,669

(22) PCT Filed: May 2, 2013
(86) PCT No.: PCT/EP2013/059200

§ 371 Date: Oct. 29, 2014

(30) Foreign Application Priority Data
May 2, 2012 (EP) 12166488.2

(51) Int. Cl. C07K 1/24; B01D 43/00; C07K 14/415; C25B 3/00

(52) U.S. Cl. CPC C07K 1/24; C25B 3/00; B01D 43/00; C07K 14/415
USPC 530/370; 205/757

(57) ABSTRACT

A method for concentrating plant proteins from an aqueous liquid is provided in which gas bubbles are generated in the liquid from a gas containing a hydrogen gas and a foam is thereby formed in which plant proteins are concentrated and extracted as a result. The gas bubbles are generated electrochemically in an advantageous manner. The method is particularly suitable for obtaining native proteins from a liquid such as potato fruit water with a very low glycol alkaloid content.

The invention relates to a process for enrichment, recovery or removal of plant proteins from an aqueous liquid and the use of hydrogen gas or a gas mixture containing hydrogen gas in a process for the recovery of plant proteins from a liquid, for purification of a liquid or depletion of plant proteins from a liquid.

The potato contains proteins with a very well-balanced amino acid composition, which is similar to the composition of chicken egg protein. Potato fruit water, which is accumulated in large quantity during starch production, contains about 0.5 to 2.5 percent by weight proteins. There is a high interest to recover proteins from such liquids for utilization.

Separation and isolation of proteins from potato fruit water without change or denaturation is difficult. Fresh potato fruit water is a complex mixture of proteins, residual starch, minerals, toxic glycoalkaloids and monomeric or polymeric, highly reactive phenolic compounds. The air oxidation of these phenol compounds causes a rapid coloration of the potato fruit water, its colour changes to a colour between brown and black. During this oxidation process the proteins may react, get linked and form undesirable byproducts.

Processes are common, which achieve the separation of the proteins by precipitation after coagulation. Coagulation is effected by heat and/or shift of pH. Such a method is described for example in DE102006050620 A1. Such processes yield only denatured proteins, which are inapplicable for nutrition and are therefore unsuitable for food production and pharmaceutical applications. In addition, possible added auxiliary reagents such as organic acids or salts have to be removed. The proteins obtained in this way lose useful functional properties by denaturation, such as solubility, ability to emulsify, ability to foam, ability to bind water and to gel thermally. More gentle processes are membrane processes (ultrafiltration), which have a high degree of efficiency and are already used for other technical purposes. However, these methods are labour-intensive.

Another method for recovering protein from potato fruit water is described in DE660992 C1. With the aid of a gas such as carbon dioxide foam is generated, in which the protein is enriched and can be removed with the foam. Such process for the enrichment and isolation of surface-active substances, including proteins, is described in DE960239 C1. Methods with an enrichment of dissolved surface-active compounds in a foam are known as “adsorptive bubble separation” (in German “Zerschäumung” or “Zerschäumungsanalyse”).

Potato protein obtained by the mentioned methods contains usually an intolerable amount of glyco alkaloids, which excludes its utilization for food.

Therefore, a process is desired, which allows the recovery of native proteins without toxic impurities.

The high content of organic matter in wastewater from the processing of plants and parts of plants, especially from potato starch production, is a problem. The practice of waste management by utitilization in animal feeding or by spray irrigation on farm land is not longer acceptable. Therefore, in potato processing and other processes there is a demand to find ways for reduction of the organic load in wastewater and of the amount of waste water.

The object of the invention is therefore to provide an alternative process for the production of pure, non-denatured, native proteins from liquids containing plant proteins, in particular from process waters and wastewaters from the processing of plant products. A further object is to provide an alternative method for the reduction of the amount of organic matter in a waste water or process water of plant processing.

The object is achieved by a process with the features described in claim 1.

The process is used for the enrichment, recovery or removal of plant proteins from an aqueous liquid containing dissolved or emulsified plant proteins. In the process gas bubbles are generated in the liquid by electrochemical gas evolution or by means of hydrogen gas or a hydrogen gas containing gas mixture. In the preferred process, foam is created containing dissolved plant proteins. Plant proteins are enriched in the foam. The foam is used for extracting soluble, non-denatured plant proteins.

In general, for obtaining plant proteins or a product with plant proteins or for purification of the liquid, foam is separated from the liquid with an appropriate device and is transferred, e.g. by discharging or skimming of foam. Advantageously, a device comprising a tube, a tubular structure or a hollow body having an input opening and an output opening is used, in which the formed foam can rise. The device is for example an electrolytic cell or an electrode compartment of an electrolysis cell, which is connected to a shaft, a conduit, a tube or tubular structure, or comprises a region in which the formed foam can rise. The tube or the analogous structures may be advantageously dimensioned in such a way that its diameter measures three times to one-tenth of the diameter of the electrolysis cell. Foaming and discharge of foam (separation of foam from the liquid in the device) results in a depletion of plant proteins of the liquid, which is in the vessel or in the container where the foam is generated. A discharge of foam (foam separation) may be effected by an overflow. The depletion of protein is also used for purification of the liquid. A foam treatment of a liquid comprises foaming and foam discharge.

In the present invention the term “plant protein” refers to any protein that is of plant origin, i.e. protein that is contained in plants, parts of plants or other plant products. Plant products are e.g. fruit, fruits, berries, nuts, pips of a plant, seeds, tubers, roots. Liquids containing dissolved plant proteins are formed for example in processing starch containing plant products, such as cereals and potatoes. Liquids containing dissolved plant proteins are formed in particular in the processing of agricultural crops or plants or their products. Agricultural crops or their plant products are for example, maize, rice, barley, wheat, rye, oats, millet, soy, tapioca, Jerusalem artichokes, potatoes and sweet potatoes, asparagus, salsify, beets, cassava, manioc, tannia, canna, yam, arrowroot (maranta), coco yam, taro, emmer, rape, cucumbers, melons, pumpkins, nuts, peanuts, sunflower seeds, sugar cane, sugar beet, carrot, cabbage, kohlrabi, apples or grapes.

Within the scope of the present invention the term “protein” stands for any molecule which comprises at least ten amino acids. Therefore, the term “protein” also comprises peptides, oligopeptides and polypeptides.

Liquids containing dissolved plant proteins are in particular plant fruit waters, e.g. potato fruit water. The term “plant fruit water” stands for any liquid containing dissolved plant material or dissolved plant substances. The term “plant fruit water” comprises process water from processing plants, parts of plants or plant products. Plant fruit water can be produced from plant waste material from agricultural crops or plants or plant products therefrom, e.g. plant peels or plant waste materials, especially potato peels. Plant fruit water can be obtained by pressing, extraction or by washing of plant material, in particular cut or crushed plants, parts of plants or plant products. Water is the preferred solvent, extraction medium or washing liquid. The water may contain additives or other solvents. However, any solvent can be used, which is known in the art as suitable for the purpose according to the invention. Further, plant fruit water can be prepared by any method known in the art as being suitable.

The fluids used contain for example 0.01 to 10 percent by weight proteins, in particular 0.1 to 5 percent by weight proteins or 0.1 to 3 percent by weight proteins. Liquids with a low protein content are usable in the process. Typically liquids with a protein content in the range of 0.5 to 3 percent by weight proteins or protein are used.

In some cases a dilution of the liquid may be advantageous. For example, the liquid is diluted with water 1:5 or 1:10. A dilution of the liquid can improve the purity of the product of the foam treatment.

The liquid, in particular plant fruit water, e.g. potato fruit water, may contain a stabilizing agent. For example a stabilizing agent may be added to potato fruit water to avoid chemical changes such as degradation reactions during storage. The use of hydrogen gas or hydrogen gas-containing gas mixtures is particularly advantageous in processes with enrichment of dissolved plant proteins from aqueous media, in particular from protein-containing solutions.

A process is called “adsorptive bubble separation” (in German “Zerschäumung”), where in at least one step a formation of foam in an aqueous liquid, containing dissolved surface active substances such as proteins, is used for producing a foam, which is enriched with these substances.

The treatment, separation and processing of the foam obtained in the process according to the invention can be conducted as in the conventional procedures for adsorptive bubble separation. The foam in an adsorptive bubble separation is formed for example by feeding a gas into the liquid. Fine bubbles may be formed by saturation of the liquid with the gas under pressure and subsequent relaxation, i.e. by depressurization. In general the formed foam is separated off, removed or discharged. The process can be operated in a batch process or continuously. It can serve for depletion of dissolved plant proteins from a liquid, for enrichment of plant proteins or for the isolation of plant proteins. Advantageously the foam is allowed to rise in a tube. A foam column is formed, which has an influence on the distribution of the plant protein and may effect a separation between different plant proteins. As the gas bubbles in the foam column have adsorptive properties, the process is called adsorptive bubble separation. Processes of adsorptive bubble separation are described for example in DE327976 C1, DE660992 C1 and DE960239 C1, which are incorporated by reference. The treatment, separation and processing of the foam in the process according to the invention may be conducted as in the conventional process of adsorptive bubble separation.

The use of hydrogen gas or hydrogen gas-containing gas mixtures facilitates product separation and allows a more gentle product recovery. Very small gas bubbles can be produced with hydrogen gas, especially by cathodic hydrogen evolution, which leads to a more effective accumulation of plant proteins in the resulting foam. From preliminary results it is concluded that gas bubbles of hydrogen exhibit favorable adsorptive properties at the phase boundary between liquid and gas. Further, a more gentle separation of plant proteins makes hydrogen gas and hydrogen gas-containing gas mixtures attractive as foam-producing gas in foaming processes for enrichment or extraction of plant proteins such as adsorptive bubble separation. The reductive conditions during foaming with hydrogen gas protect the products and the substances in the liquid against atmospheric oxygen. This is particularly advantageous for potato fruit water, which contains substances with phenolic groups sensitive to oxidation (e.g. polyphenols), whose oxidation products make a work up of the liquid more difficult and cause a blackening of the liquid. Therefore, the product obtained by adsorptive bubble separation with hydrogen gas contains less impurities from oxidative degradation or decomposition. Unstable proteins are recovered without damage. The use of hydrogen gas or hydrogen gas-containing gas mixtures for the enrichment and isolation of plant proteins by foaming is therefore particularly advantageous for the recovery of native proteins. In general the recovered proteins retain the ability to dissolve in water.

The use of hydrogen gas for foaming can be combined with one or more different gases, simultaneously or sequentially. For example, the gases nitrogen, oxygen, carbon dioxide may be used in addition to hydrogen. Inert gases or reactive gases may be used in addition. For example, foaming may start with the use of hydrogen gas and may be continued using another gas.

Liquid products containing plant protein, which were obtained by foaming, may be further enriched by additional (single or multiple) foam treatment (adsorptive bubble separation).

Processes with foaming by electrochemically generated gases or gas mixtures in the liquid are of advantage, in particular electrochemically generated hydrogen gas or electrochemically generated gas mixtures containing hydrogen gas. Gas bubbles electrochemically (electrolytically) generated in the liquid are different from conventionally produced gas bubbles in adsorptive bubble separation. Electrolytically generated gas bubbles are in general much smaller than generated with a gas sparger and usually lead to a foam consistency favorable for enrichment and separation of dissolved plant proteins.

In principle, cathodic hydrogen evolution and anodic oxygen evolution may be used for gas bubble generation. However, hydrogen gas and oxygen gas differ in the adsorption behavior. Surprisingly it has been found that foam with a low content of glycoalkaloids is obtained, when hydrogen gas bubbles are generated electrolytically for protein recovery from potato fruit water. Hydrogen gas can therefore be used for liquids with organic toxic contaminations for production of plant protein products with reduced content of toxic impurities. In addition, hydrogen gas protects against oxidative reactions, as has already been mentioned. Foaming with hydrogen gas leads to a better product quality of recovered plant proteins. A cathodic hydrogen evolution is preferred for foam generation, especially using a divided electrochemical cell.

Therefore, another object of the invention is a process for the recovery or removal of plant proteins from an aqueous liquid, where electrochemically gas bubbles of hydrogen gas or a gas mixture containing hydrogen gas are generated in the liquid and a foam containing dissolved plant proteins is formed, wherein the foam or a part of it is separated from the liquid and a product or intermediate with plant proteins is obtained having a low content of toxic impurities.

The process is particularly advantageous with potato fruit water as liquid or with a liquid containing potato proteins. Here a product or intermediate product with plant proteins is obtained, which has a low content of glycoalkaloids or contains less than 100 ppm glycoalkaloids, preferably less than 50 ppm of glycoalkaloids, more preferably less than 10 ppm of glycoalkaloids (1 ppm is a gram per 10⁶ grams).

The process for recovery or removal of plant proteins from an aqueous liquid is carried out without precipitation of plant proteins. The process variant with electrochemical gas bubble formation in the liquid is no electroflotation, since electroflotation is always used for separation of particles.

The electrochemical cell (electrolysis cell) may be a divided or an undivided cell. Preferably, the electrochemical cell comprises at least one tube or tube-like structure, wherein the formed foam can rise. The tube or tube-like structure is generally vertically arranged on the electrochemical cell or connected to the electrochemical cell. Generally an electrochemical cell is advantageous, which comprises a hollow member or a hollow structure or is connected to a hollow part with an upper and lower opening, wherein formed foam can rise.

The tube or tube-like structure, in which formed foam can rise, has for example a length of at least 0.5 m, advantageously at least 1 m, more advantageously at least 2 m. The upper end of the tube or tube-like structure is formed for example as an overflow. The length of the tube or tube-like structure has an influence on the consistency and composition of the overflowing foam.

Sample fractions (foam samples) can be collected from the overflow for a fractionated plant protein recovery. From the overflow discharged foam is further processed for plant protein isolation. In general, in a first step foam is transformed into collapsed foam. For example, overflowing foam is fed to a foam breaker (device for getting collapsed foam) or into a collecting container. The foam is turned into a liquid, which has a higher protein content than the initial liquid. This liquid product (protein solution) can be further processed for isolation of plant protein or for obtaining a plant protein concentrate. Suitable processes for work up of such protein solutions are known in the art.

Usually very small gas bubbles of hydrogen are formed during the electrolytic production of hydrogen in the liquid to be treated. The formed gas bubbles generally have a diameter of less than 50 μm. Gas bubbles with a diameter of less than 30 μm, in particular below 25 μm, can be prepared in a simple manner. The electrochemically generated gas bubbles, preferably generated in the treated liquid, preferably have a diameter in the range of 1 to 50 μm, more preferably in the range 1 to 30 μm, in particular in the range 5 to 25 μm. Small gas bubbles are very beneficial. They improve, among other things, the protein enrichment in the foam.

Generally, for the electrochemical generation of hydrogen gas electrodes with electrode materials are used as cathode, which are suitable for cathodic hydrogen evolution. They contain for example platinum, nickel, iron, iridium, ruthenium, palladium or conductive carbon-containing materials. Cathode materials are for example platinum, glassy carbon, graphite, conductive diamond, in particular boron-doped diamond (BDD), or conductive carbonaceous materials, in particular carbon nanotubes and carbon black. Platinum cathodes are for example platinum-coated titanium electrodes. For example, graphite is used as electrode in the form of flat structures (e.g. plates), a planar micro-structured graphite layer, formed parts or particles. A graphite-particle-electrode is for example a graphite particle bed. The particle diameter of a graphite particle bed ranges for instance between 0.1 mm and 5 mm. The particulate electrode can be used as a fixed bed or a fluidized bed electrode. Carbonaceous cathode materials such as graphite, conductive diamond and carbon electrodes are advantageous.

The electrolyte, in particular the catholyte (electrolyte in the cathode compartment), preferably has a pH value in the range of 4 to 9, more preferably 4 to 8.8, further more preferably from 4 to 8.5, even more preferable from 4.5 to 8.2, particularly preferably from 4.5 to 8.0 and most preferably from 4.8 to 6.5. The liquids that contain plant proteins can be generally used directly as electrolyte. The liquid used, e.g. potato fruit water, usually has a pH value in the range of pH 4 to pH 7. The pH value of the liquid may be adjusted to the desired value.

The pH value of the electrolyte, in particular of the catholyte, can be kept constant or can be changed during the electrolysis. In many cases the pH remains constant in batch operation using an undivided cell. A pH gradient during foaming can be realized in a simple way by using the pH drift during electrolysis in a divided cell with batch operation, as the catholyte gets more alkaline during electrolysis.

The amount of gas, the amount of gas bubbles and gas flow rate can be adjusted by changing the current density at the cathode.

Advantageously one or more divided electrolytic cells are employed, which can be operated discontinuously (batch operation) or continuously (flow operation). The divided electrolytic cell may be in particular a flow cell.

The electrolysis is generally carried out at a temperature in the range of 0° C. to 40° C., preferably in the range of 10° C. to 30° C., in particular in the range of 15° C. to 30° C. or in the range or 15° C. to 25° C. For instance, the electrolysis is carried out at room temperature.

Advantageously the electrolytic cell is dimensioned in such a way, that the height of the electrolytic cell exceeds the width or diameter of the electrolysis cell many times. Advantageously electrodes are used with a height-to-width ratio (for flat electrodes) or height-to-diameter ratio (for cylindrical electrodes) greater than 1. Very advantageous is a height-to-width ratio or height-to-diameter ratio of the electrodes of at least 10. Such dimensions of the electrodes create a longer way for gas bubble movement along the cathode.

In the case of a continuously operated, divided electrolytic cell the cathode and anode compartment are usually supplied with electrolyte from tanks by means of pumps. Operation with a circular flow between tank and electrode compartment is of advantage. In general liquids such as plant fruit water, in particular potato fruit water, are used as catholyte. Such fluids, in particular after foam treatment and protein removal in the cathode compartment, are preferably used as anolyte. It may be advantageous to use other fluids as anolyte, depending on the anode reaction chosen.

For instance, the cathode reaction is carried out at a current density of 5 to 15 mAcm⁻². In batch operation the electrolysis time is 2 to 3 hours as an example.

Anodic hydrogen oxidation may be an alternative to anodic oxygen evolution or anodic oxidation of organic matter. A combination of a cathodic hydrogen generation for foam production with an anodic oxidation of hydrogen offers advantages in view of energy demand. Preferably a divided cell is used. Particularly advantageous for the anodic oxidation of hydrogen are gas diffusion electrodes. For example, a suitable gas diffusion electrode is prepared by pressing a mixture of platinized carbon black and polytetrafluoroethylene on a cation exchange membrane, where a 40 to 50 μm thick coating on the cation exchange membrane is generated. Such gas diffusion electrodes and the anodic oxidation of hydrogen are described in EP0800853 A2, which is hereby incorporated by reference. Combining cathodic hydrogen generation for foaming with an anodic oxidation of hydrogen can reduce the energy consumption significantly.

Therefore, another object of the invention is a process, wherein hydrogen gas is generated at the cathode for a generation of foam in a liquid with plant proteins, e.g. process water or wastewater, in an electrochemical cell, preferably a divided cell, and hydrogen is oxidized at the anode as counter-reaction.

Advantageously the hydrogen gas produced electrolytically is used for treatment of a fluid in or outside of the electrolytic cell, where the cathodic generation of hydrogen gas bubbles generally takes place in the liquid to be treated under foaming. For example, electrochemically generated hydrogen gas can be removed from the electrolysis cell and can be fed back in the form of fine gas bubbles into the electrolytic cell or the cathode compartment respectively or into an external tank with liquid to be treated (adsorptive bubble separation outside the electrolytic cell). The injection or feeding with hydrogen gas can be performed while the electrolysis is running or turned off. For injection or recirculation of hydrogen gas a gas bubble generator may be used. Generally every device, which is deemed suitable to the skilled person, may be used as gas bubble generator. Suitable devices are frits made of metal or glass, which release the gas bubbles, or injectors, which may be directed against a baffle plate, or Venturi injectors. The injectors may also be combined with a static mixer, which dissipates the gas flow from the injector into small gas bubbles and distributes them as homogeneous as possible. Advantageously pressure release can serve for gas bubble generation. When pressure release is used, hydrogen gas needs no recirculation.

Anodic hydrogen oxidation is preferred to recycling or reuse of hydrogen gas, in particular of electrolytically generated hydrogen gas for foam treatment of a liquid containing dissolved plant proteins in or outside of an electrochemical cell, since the electrolytic gas bubble generation is superior to other methods for gas bubble generation.

Hydrogen gas may be generated by electrolysis continuously (e.g. galvanostatically or potentiostatically) or discontinuously in the process of foam formation for enrichment, recovery or removal of dissolved or emulsified plant proteins in a liquid. For example, hydrogen gas is generated in pulses, preferably in the liquid. In such a pulse operation the current is repeatedly turned on and off for a certain period of time. Such current pulses can be of constant duration or variable in duration. Advantageously the sequence of current pulses is produced by means of a control device or a control unit. The control device may be e.g. a pulse generator or a programmable device. The length of the current pulse can be controlled or sensor controlled. For example, the electrolysis current can be interrupted, when the foam formation comes to a certain size or the liquid gets a certain property. Such control variables are for example, the height of foam in a tube, optical properties such as transparency or conductivity of the liquid. The control of the electrolytic current may be performed with the aid of optical sensors (e.g. photocell, photodiode, phototransistor, photoresistor) or electrical sensors (conductivity sensors, capacitance sensors) or other suitable sensors.

In the process of foam formation for enrichment, recovery or removal of dissolved or emulsified plant proteins in a liquid the current or current density may be changed during the electrolytic hydrogen gas generation. In a continuous electrolysis the current may be increased, decreased or varied. The current density may be controlled during electrolysis. The control can be as described for pulsed operation. The variation of the current can be combined with a pulse operation. For example, the current density of current pulses may be varied, e.g. a sequence of pulses of constant current with variable height from pulse to pulse or a sequence of pulses of variable current with constant height from pulse to pulse or a sequence of pulses of variable current with variable height from pulse to pulse.

The electrodes may get contaminated or covered with deposits during the electrolysis. Therefore, it can be advantageous to change the polarity of the electrode once or multiple times during electrolysis. For an electrolysis with reversal of the polarity of the electrodes an electrolytic cell with suitable electrodes or with a symmetrical structure (same electrodes) is advantageous. For example, a divided or undivided electrolytic cell with platinum electrodes for cathodic hydrogen evolution and anodic oxygen evolution with anodic oxidation of organic matter is suitable for reversing polarity.

Another object of the invention is a process for enriching, recovery or removal of plant proteins from an aqueous liquid, where hydrogen gas is produced electrolytically and electrolysis is performed continuously or discontinuously, at a constant or variable current, controlled, regulated or program controlled, pulsed, with current pulses or with reversing the polarity of the electrodes.

Conducting the process for protein recovery, 80 to 90 percent of the protein content of the treated liquid, e.g. potato fruit water, are removed. Surprisingly the proteins respectively the product extracted from plant fruit water, in particular potato fruit water, contain very low amounts of glycoalkaloids. In this aspect, the process is superior to the known processes. The isolated proteins are soluble. This makes the products obtained by foaming with hydrogen gas very suitable for applications in the field of food production.

The removal of plant proteins of a liquid by foaming, in particular with electrochemically generated foam, can be used for purifying liquids from processes, where plant products are processed.

In a process for the purification of liquids containing organic matter and plant proteins, such as process water, wastewater or plant fruit water, advantageously the content of organic matter of the liquid is reduced by foam treatment and the obtained depleted liquid is used for further purification. The further purification of the depleted liquid is performed, for example, by anodic oxidation. Advantageously, the depleted liquid is used as electrolyte, in particular as anolyte, in an electrolytic cell. An extraction of plant proteins from a liquid with plant proteins by foaming with cathodic evolution of hydrogen (cathodic foam treatment) and a purification by anodic oxidation of organic components of a liquid, e.g. depleted liquid from an adsorptive bubble separation or from foam treatment, can advantageously be carried out in parallel in a divided electrolysis cell or less preferably in an undivided electrolysis cell.

Another object of the invention is a process, wherein a cathodic foam treatment of a liquid and a purification of another liquid (e.g. a depleted liquid) by a direct or indirect electrochemical oxidation are combined.

A direct electrochemical oxidation is an anodic oxidation. An indirect electrochemical oxidation is an oxidation by an oxidant, which is formed at the anode, e.g. a redox mediator. The use of a redox mediator requires a divided electrolytic cell. Advantageously, the anode is an electrode with conductive diamond, e.g. a BDD-coated electrode.

By cathodic foam treatment and subsequent or parallel anodic oxidation the COD and BOD value of a liquid containing plant proteins can be strongly reduced (COD stands for Chemical Oxygen Demand, BOD stands for biological oxygen demand).

A divided cell is generally used for the purification process (combined process). Preferably an ion exchange membrane separates the electrode compartments. An electrode suitable for hydrogen evolution is used as cathode. An electrode is preferred as anode, which is made of an electrode material having a high oxygen overvoltage such as an electrode material containing SnO₂, smooth platinum or conductive diamond, like BDD. Preferred as anode is an electrode made of a conductive substrate (Nb, Ti, corrosion resistant metal or metal alloy; metal substrates) in the form of expanded metal, coated with boron-doped diamond. The process uses for example a flow cell. In this case, the liquid, e.g. fresh potato fruit water, is fed into the cathode compartment (catholyte) and depleted liquid (used catholyte) is fed into the anode compartment. This can be done by means of a pump from a reservoir in each case. The use of a divided cell with ion exchange membrane and a “zero-gap” arrangement of electrodes (on both sides of the ion exchange membrane directly arranged electrodes such as grid electrodes) is very advantageous.

For example, a graphite plate serves as cathode and an electrode coated with conductive diamond (e.g. BDD, boron-doped diamond) as anode. Advantageously a tube is tightly arranged over the cathode compartment (e.g. 0.3 m, 0.5 m, 1 m or 2 m long), wherein formed foam can rise. In a process for reducing the content of organic matter of a liquid (process for purifying a liquid), where the liquid is treated cathodically and anodically, the pump speed for the catholyte is for example 5 ml/min and for the anolyte for example 1 l/min. In the reservoir with circulated anolyte e.g. potato fruit water or cathodically depleted potato fruit water as anolyte usually a pH of less than 3 is obtained soon. Here organic matter precipitates and the solution clarifies. In a flow-process with cathodic and anodic treatment of a liquid such as plant fruit water, the flow rates of the electrolytes are advantageously adjusted such, that the residence time of the anolyte in the anode compartment is less than the residence time of the catholyte in the cathode compartment. Very advantageous is a residence time of the anolyte in the anode compartment five to ten times, preferably ten times, smaller than the residence time of the catholyte in the cathode compartment.

### EXAMPLES

### 1. Protein Extraction with Electrolysis Cell in Batch Mode

The electrolytic cell contains an electrode stack with cathode and anode grid electrodes, separated by plastic grids as spacers (distance d=1 mm). The cathodes are made of stainless steel meshes, the anodes are expanded metal electrodes made of niobium coated with boron-doped diamond. The vessel of the electrolytic cell is in the simplest case a glass beaker with a volume of 2.5 liters. The effective area of a single electrode is 120 cm². The electrode stack is made of two anodes and three cathodes, alternating. The entire cathode surface is 360 cm².

1.8 liters of potato fruit water are filled in and are moderately stirred with a magnetic stirrer. The batch electrolysis was carried out at an initial temperature of 15° C. and a final temperature of 22.5° C. after 10 hours of operation. The constant current was 3.6 A and the average cell voltage was 1.7 V. The pH value was adjusted to pH 5 and kept constant. For workup, the protein foam that formed on the surface of the liquid was skimmed off and dried in rotary evaporator under partial vacuum. 4.5 g of a whitish-yellow to pale yellow powder was obtained, which was nearly all protein as determined by the nitrogen assay according to Kjeldahl.

### 2. Electrolysis with Reversal of Electrode Polarity

A simple electrolytic cell was used: a glass beaker (graduated volume: 800 ml, height: 14 cm, diameter: 11 cm) as a vessel for electrolysis and two electrodes of expanded and platinized titanium metal (height: 6 cm, width: 10 cm) as cathode and anode.

In this example the distance of migration of bubbles through the solution is determined solely by the electrode height. The height of formed foam above the liquid is 3.5 cm.

Potato fruit water with a content of 1.2 percent by weight (% w/w) protein (according to Kjeldahl assay) is used as electrolyte. This solution is slowly pumped with a peristaltic pump from a reservoir (volume: 5 liters) into the beaker and removed with the same pump, so that the liquid surface is maintained 1 cm above the electrodes.

The electrodes are connected to a variable DC source. The electrolysis is carried out at a constant current of 0.9 A. The initial cell voltage is 1.5 V. Theoretically an electrolysis time of 83 hours is required with these settings.

The pH was adjusted to pH 5 and kept constant.

After 20 hours of operation the voltage increased by 250 percent. The reason is a beginning blockage of the electrodes (formation of a contaminant layer on the electrodes). As a counter measure the electrodes were reversed now for 2 minutes every 0.5 hours. After 3 hours the voltage dropped to 130 percent of the initial value and remained constant during the residual operating time.

After an operating time of 50 hours the DC source is disconnected from the electrodes and the weight of the collected foam and its protein content (by an adapted Kjeldahl assay) are determined. The electrolyte was analyzed before and at the end of the electrolysis with the Kjeldahl assay.

In this experiment an enrichment factor of 6.5 (ratio of protein content in the foam to protein content of the original liquid) was found, which means that the protein is concentrated 6.5 times in the foam. The analytical results indicate that 60 percent of the protein of the liquid was recovered with the foam.

### 3. Electrolysis Cell with Channel for Rising Foam

The electrolysis cell consists essentially of a shaft (height: 100 cm; base area: 0.12 cm width, 5.5 cm depth), wherein expanded titanium metal electrodes, fixed in a frame at a distance of 2 cm, are placed. The expanded metal anode is coated with a mixture of oxides of noble metals. The cathode is uncoated. The electrode area is 275 cm² respectively.

After filling the electrolysis cell with the liquid a free channel of about 0.5 m in length remains, in which the foam can rise up. The mixing of the electrolyte is achieved by pumping (peristaltic pump) electrolyte from the upper region to the lower region of liquid in the electrolytic cell.

During operation the foam rises, depending on its consistency, about 3 to 10 cm over the edge of the vessel (upper end of the shaft) until foam breaks off at the top. This foam is collected in a trough and dried at 25-30° C. under low pressure.

A volume of 3.4 liters of potato fruit water is filled into the electrolysis cell. The electrodes are connected to a DC current supply. A constant current of 2.2 A flows, which corresponds to a current density of i=8 mA/cm². After an electrolysis time of 4.7 hours, 1.17 g of a powder is obtained from the collected foam after vacuum drying, as described above, corresponding to a protein content of 88 percent (determined by the Kjeldahl assay).

### 4. Foaming with Hydrogen Gas in a Divided Cell

An electrolytic cell (closed system) is used, which is divided by a cation exchange membrane into a cathodic and an anodic compartment, and is equipped with platinum electrodes. The anodic compartment is filled with dilute acetic acid. The cathodic compartment is connected to a bottom outlet of a storage vessel filled with potato fruit water of pH 5. A tube extends from the upper portion of the electrolytic cell and is so arranged that the foam transported in it can fall into a collecting vessel from above.

Circulated liquid is pumped with a peristaltic pump from the reservoir into the lower part of the cell. The gas bubbles move through the liquid (liquid column over the electrodes) passing a longer distance compared to the mere electrode height. Also the foam column formed is larger than in the previous examples. The ratio of electrode length/length of liquid column/length of foam column is about 1:3.3:5.

Identical platinum electrodes are used and a current density of 7.7 mA/cm² is applied.

The foam, which emerges from the top of the tube, is collected and analyzed as described above. The dry substance produced from the foam has a protein content of 92 percent (determined by the Kjeldahl assay).

### COMPARATIVE EXAMPLE

A glass cylinder (height: 1 m, diameter: 8 cm) is filled with 2.5 liters of potato fruit water. Air is sparged through a frit of D3 porosity at the bottom of the vessel. This results in a liquid column with a height of 50 cm and a foam column with a height of 50 cm. The gas is fed at a gas flow velocity v=30 ml/min. In contrast to the results of previous experiments the foam contained very large bubbles and had overall a very low density. The foam is difficult to handle under technical conditions in a production process. The content of dry matter is very low. The foam had a protein enrichment factor of 1.3.

1. A process for the enrichment, recovery or removal of plant proteins from an aqueous liquid, said process comprising generating gas bubbles of hydrogen gas or a hydrogen gas containing gas mixture or electrochemically generated gas bubbles in the liquid, funning a foam that contains dissolved plant proteins, and separating the foam from the liquid.

2. A process according to claim 1, wherein said process comprises generating the gas bubbles electrochemically in the liquid in an undivided or divided electrolytic cell.

3. A process according to claim 1, wherein said process comprises generating the gas bubbles on at least one electrode containing platinum, iridium, ruthenium, iridium and ruthenium, graphite, conductive carbon material or conductive diamond.

4. A process according to claim 1, wherein said process further comprises recovering a liquid product or intermediate product from the foam separated from the liquid, which contains a higher content of plant proteins than in the initial liquid.

5. A process according to claim 1, wherein said process further comprises generating the gas bubbles within a device which comprises at least a tube, a tubular structure, a hollow body having an inlet opening and an outlet opening or a region in which the formed foam can rise, and the device is an electrolytic cell or a device with an entity for gas bubble generation.

6. A process according to claim 1, wherein said process further comprises producing a liquid depleted of plant proteins from the liquid, which is thereafter subjected to a direct or indirect electrochemical oxidation.

7. A process according to claim 1, wherein said process further comprises generating gas bubbles in a divided electrolytic cell cathodically in the liquid to remove plant proteins and oxidizing anodically a liquid depleted from plant proteins or a liquid containing organic substances via a direct or indirect electrochemical oxidation.

8. A process according to claim 1, wherein the liquid is a process water or wastewater from processing of plants, parts of plants or plant products or the liquid is plant fruit water or potato fruit water.

9. A process according to claim 1, wherein said process comprises producing the gas bubbles electrochemically in the liquid in an undivided or divided electrolytic cell, where hydrogen gas is generated at the cathode and oxygen gas at the anode or hydrogen gas is generated cathodically and hydrogen gas is oxidized anodically.

10. A process according to claim 1, wherein the liquid has a pH value in the range of pH 4 to pH 7 at the beginning of the generation of gas bubbles and the pH of the liquid is kept constant or changes.

11. A process according to claim 1, wherein said process comprises producing the gas bubbles electrochemically by electrolysis and operating the electrolysis continuously or discontinuosly, pulsed or with at least one reversal of the polarity of the electrodes.

12. A foam containing dissolved plant proteins produced by generating gas bubbles with hydrogen gas or a gas mixture containing hydrogen gas in a liquid containing plant proteins and separating formed foam from said liquid.

13. A foam according to claim 12, wherein the hydrogen gas or the hydrogen gas containing gas mixture is produced electrochemically in the liquid.

14. A foam according to claim 12, wherein the liquid is a plant fruit water or potato fruit water.
